(19) United States
(12) Patent Application Publication
(10) Pub. No.: US 2011/0021776 A1
(43) Pub. Date: Jan. 27, 2011

(54) COMPOSITIONS FOR THE TREATMENT OF CENTRAL NERVOUS SYSTEM DISEASES AND DISORDERS

(75) Inventors: Irina Shcherbakova, Midvale, UT (US); Yuri Nikolyukin, Moorsetown, NJ (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: MediProPharma, Inc., Midvale, UT (US)

(21) Appl. No.: 12/884,424

(22) Filed: Sep. 17, 2010

Related U.S. Application Data
(63) Continuation-in-part of application No. 11/890,166, filed on Aug. 3, 2007, now Pat. No. 7,820,691.
(60) Provisional application No. 60/840,596, filed on Aug. 28, 2006.
(60) Provisional application No. 61/266,671, filed on Dec. 4, 2009.

(51) Int. Cl. C07D 471/04

(52) U.S. Cl. 546/70

(57) ABSTRACT

Compositions and methods for treating, preventing and/or delaying the onset and/or the development of diseases and disorders of the central nervous system are disclosed. The present disclosure relates to indoloquinoline compounds that are capable of inhibiting at least one protein kinase, and to methods for preparing and uses of such compounds. The compounds described herein are administered to patients to achieve a therapeutic effect.

### RELATED APPLICATIONS

This application is a continuation-in-part of U.S. application Ser. No. 11/890,166, titled INDOLOQUINONLINE COMPOUNDS AS CALCIUM CHANNEL BLOCKERS, filed on Aug. 3, 2007, which claims priority to U.S. Provisional Application No. 60/840,596, filed Aug. 28, 2006. This application also claims priority to U.S. Provisional Application No. 61/266,671, filed Dec. 4, 2009. Each of the foregoing applications is hereby entirely incorporated by reference herein.

### TECHNICAL FIELD

The present disclosure relates to compositions and methods for treating, preventing and/or delaying the onset and/or development of diseases and disorders of the central nervous system. More specifically, the present disclosure relates to indoloquinoline compounds that are capable of regulating phosphorylation processes through the inhibition of at least one protein kinase, DYRK1A, and to methods for preparing and using such compounds. The compounds described herein are administered to patients to achieve a therapeutic effect.

### BRIEF DESCRIPTION OF THE DRAWINGS

FIG. 1 illustrates the reduction of tau phosphorylation at pSer262/pSer356 (12E8), pThr231 and pSer396 sites by the compounds of Examples 1 and 2 after a 96 hr in vitro treatment of human H4 neuroglioma cells, and levels of total tau protein, DYRK1A and tubulin vs control values in Western blotting (A) and quantitative graphs (B).

### DETAILED DESCRIPTION

Protein kinases with a conserved catalytic domain make up one of the largest superfamilies of eukaryotic proteins, and play many key roles in biology and disease. Protein kinases, which serve critical functions in signaling pathways, are useful therapeutic targets. Approximately eight protein kinase inhibitors have been recently approved by the FDA in the United States.

Evolutionally conserved families of protein kinases known as dual-specificity tyrosine phosphorylation-regulated kinases (DYRKs) are involved in diverse biological functions. The DYRK family comprises several members in mammals, of which DYRK1A and DYRK1B are predominantly localized to the nucleus. Expression of DYRK1A is detected in several regions of the central nervous system (CNS), from development to adulthood, especially in the cortex, hippocampus and cerebellum. The human DYRK1A gene has been implicated in the pathogenesis of Down syndrome due to its location in the Down syndrome critical region on chromosome 21, which is present in three copies in Down syndrome patients.

Phosphorylation by protein kinases is a major mechanism by which virtually every activity of eukaryotic cells is regulated. DYRK1A has been shown to promote pathophysiological hallmarks of neurodegenerative diseases and disorders via direct phosphorylation of the most abundant components, such as tau in neurofibrillary tangles, α-synuclein in Lewy bodies, amyloid-β precursor protein, and septin 4. The levels and catalytic activity of DYRK1A are increased in neurons of the CNS when they undergo degeneration.

Tau is the major neuronal microtubule-associated protein (MAP) which promotes assembly and stabilizes microtubules. Tau functions as a microtubule organizing protein that increases microtubule stability by suppressing dynamic instability. Hyperphosphorylation of the tau protein leads to the formation of paired helical filaments (PHFs) and neurofibrillary tangles (NFTs), microtubule instability and functional loss of the microtubule cytoskeleton, contributing to neuronal cell dysfunction and cell death. Alterations in the interaction of tau protein with microtubules and their stabilization have been identified in certain neurodegenerative diseases and disorders, and are called tauopathies. The extent of tau phosphorylation is regulated by the activities of various protein kinases and phosphatases. Increased activity of these kinases or decreased activity of these phosphatases lead to hyperphosphorylation of tau, which results in the formation of PHFs and NFTs.

DYRK1A is a kinase that phosphorylates tau at multiple threonine and serine sites including Thr181, Ser202, Thr202, Thr217, Thr231, Ser396, Ser400, Ser404 and Ser422, both in vitro and in cultured cells. Tau hyperphosphorylation at several sites, such as Thr212, Thr231, Ser262 is confirmed to cause neurodegeneration. Inhibition of DYRK1A phosphorylation of the tau protein underlies the molecular mechanism for the disease-modifying treatment of neurodegenerative diseases and disorders associated with tauopathies.

Moreover, DYRK1A is a priming kinase that facilitates the further phosphorylation of proteins by other kinases. Phosphorylation of tau by DYRK1A primes its further phosphorylation by glycogen synthase kinase-3β (GSK-3β), inhibits tau's biological activity and promotes its self-aggregation to PTFs. Inhibition of DYRK1A may reduce tau phosphorylation by GSK-3β, thus acting at two critical kinase pathways with protection against tau hyperphosphorylation and toxicity.

Thus, research shows that DYRK1A kinase may play a pivotal role in neuronal cell death and the pathogenesis of neurological and neurodegenerative disorders and diseases associated with tauopathies. Inhibitors of the DYRK1A kinase offer a unique approach toward the disease-modifying treatment of neurodegenerative and neurological diseases and disorders.

The present disclosure features inhibitors of DYRK1A kinase. “Inhibitors of DYRK1A kinase” refers to compounds able to inhibit phosphorylation by DYRK1A kinase. The ability of a compound to “inhibit phosphorylation by DYRK1A kinase” means that the compound causes a decrease in one or more of the kinase activities evoked by DYRK1A kinase. For example, inhibition of DYRK1A may be shown by compounds exhibiting a Kd of about 5 uM or lower in a DYRK1A binding assay, about 10 uM or lower and about 50 uM or lower.

The use of inhibitors of DYRK1A kinase may achieve a beneficial effect in a patient, as described herein. More specifically, the present disclosure demonstrates the ability of the compounds, which are inhibitors of DYRK1A kinase, to achieve CNS effects. Also described herein are techniques which may be used to obtain additional compounds as inhibitors of DYRK1A kinase.

Examples of inhibitors of DYRK1A kinase, containing a 2,3,4,7-tetrahydroindolo[2,3-c]quinoline core, are provided by the chemical formula depicted in Structure I and the accompanying description.

wherein:

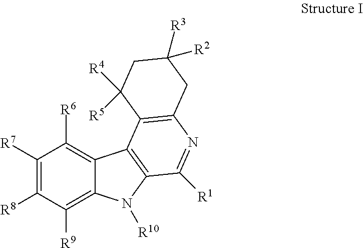

R¹ is one of: H, lower alk, cycloalk, aryl, arylalkyl. In certain embodiments, R¹ may be H or lower alkyl. In other embodiments, R¹ may be methyl. In other embodiments, R¹ may be C2-C6 alkyl. For example, R¹ may be ethyl or isopropyl. In yet other embodiments, R¹ may be one of H or alkyl, with the proviso that R¹ is not methyl.

R² and R³ are each independently selected from one of: H, lower alkyl, cycloalk, aryl, arylalkyl; or R² and R³ are together —(CH₂)ₙ— and n is 6, 5 or 4; or R² and R³ are together —CH(lower alkyl)(CH₂)ₙ— and n is 5, 4 or 3. In certain embodiments, R² and R³ may be each independently selected from one of: H, lower alkyl, wherein at least one of R² and R³ is hydrogen. In certain embodiments, R² and R³ may each be methyl. In other embodiments, R² and R³ may together be cyclohexyl. In still other embodiments, one of R² and R³ may be isopropyl or methyl.

R⁴ and R⁵ are independently one of: H, NH₂, OH or lower alk; or R⁴ and R⁵ are together O, S or NOH. In certain embodiments, R⁴ and R⁵ are together O.

R⁶, R⁷, R⁸, and R⁹ are independently one of: H, halogen, CN, CF₃, OCF₃, lower alkyl, cycloalk, lower alkoxy, NH-lower alkyl, NH-alkylaryl, N(lower alkyl)₂, C(O)OH, C(O)O-lower alkyl, OH, OC(O)-lower alkyl. In certain embodiments, R⁶, R⁷, R⁸, and R⁹ may be independently selected from one of: H, halogen, or methoxy, wherein at least one of R⁶, R⁷, R⁸, and R⁹ is halogen or lower alkoxy. In other embodiments, R⁶, R⁷, R⁸, and R⁹ may each be H.

R¹⁰ is one of: H, lower alkyl, cycloalk, arylalkyl, aryl. In certain embodiments, R¹⁰ is H;

and pharmaceutically acceptable salts, hydrates, tautomers, solvates and complexes thereof.

“Alk” refers to either alkyl or alkenyl. “Lower alk” refers to either lower alkyl or lower alkenyl.

“Alkenyl” refers to an optionally substituted hydrocarbon group containing at least one carbon-carbon double bond between the carbon atoms and containing 2 to 6 carbon atoms joined together. The alkenyl hydrocarbon group may be straight-chain. Certain straight-chain alkenyl embodiments have 2 to 4 carbons.

“Alkyl” refers to an optionally substituted hydrocarbon group joined by single carbon-carbon bonds and having 1 to 6 carbon atoms joined together. The alkyl hydrocarbon group may be straight-chain or contain one or more branches. Branched- and straight-chain alkyl embodiments may have 1 to 4 carbons, each of which may be optionally substituted. Alkyl substituents are each independently selected from the group consisting of: lower alkyl, unsubstituted aryl, OH, NH₂, NH-lower alkyl, and N(lower alkyl)₂. In certain embodiments, no more than two substituents are present. For example, alkyl may be a lower alkyl which is unsubstituted branched- or straight-chain alkyl having 2 to 4 carbons. In certain embodiments, alkyl may be a lower alkyl having 1 to 4 carbons.

“Cycloalk” refers to an optionally substituted cyclic alkyl or an optionally substituted non-aromatic cyclic alkenyl and includes monocyclic and multiple ring structures such as bicycles and tricycles. The cycloalkyl has 3 to 15 carbon atoms. In certain embodiments, cycloalkyl has 3 to 6 carbon atoms. Optional substituents for cycloalk are independently selected from the group described above for alkyl and alkenyl.

“Aryl” refers to an optionally substituted aromatic group with at least one ring having a conjugated or fused ring system. Aryl includes carbocyclic aryl, heterocyclic aryl and biaryl groups, all of which may be optionally substituted. In certain embodiments, the aryl is optionally substituted phenyl.

“Arylalkyl” refers to an aryl-(C₁-C₆)alkyl substituent wherein the alkyl group may be linear, such as benzyl or phenethyl, or branched. The alkyl portion bonds at the point of attachment to the parent molecule.

“Alkoxy” refers to oxygen joined to an unsubstituted alkyl 1 to 4 carbon atoms in length. In certain embodiments, the alkyl is 1 to 2 carbons in length. For example, the alkoxy may be methoxy.

“Halogen” refers to fluorine, chlorine, bromine or iodine. “Haloalkoxy” refers to an alkoxy substituent wherein the alkyl is substituted with at least one halogen.

The indoloquinoline compound of Structure I may contain one or more asymmetric carbon atoms and may exist in racemic and optically active forms. All of these compounds and diastereomers are contemplated to be within the scope of this disclosure.

Exemplary embodiments include indoloquinoline compounds:
3,3,6-trimethyl-2,3,4,7-tetrahydroindolo[2,3-c]quinolin-1-one;
11-fluoro-3,3,6-trimethyl-2,3,4,7-tetrahydroindolo[2,3-c]quinolin-1-one;
9-fluoro-3,3,6-trimethyl-2,3,4,7-tetrahydroindolo[2,3-c]quinolin-1-one;
10-fluoro-3,3,6-trimethyl-2,3,4,7-tetrahydroindolo[2,3-c]quinolin-1-one;
3-isopropyl-6-methyl-2,3,4,7-tetrahydroindolo[2,3-c]quinolin-1-one;
10-methoxy-3,3,6-trimethyl-2,3,4,7-tetrahydroindolo[2,3-c]quinolin-1-one;
9-methoxy-3,3,6-trimethyl-2,3,4,7-tetrahydroindolo[2,3-c]quinolin-1-one;
3,6-dimethyl-2,3,4,7-tetrahydroindolo[2,3-c]quinolin-1-one;
10-fluoro-3,3-dimethyl-6-isopropyl-2,3,4,7-tetrahydroindolo[2,3-c]quinolin-1-one;
3,3-dimethyl-6-ethyl-2,3,4,7-tetrahydroindolo[2,3-c]quinolin-1-one.
3,3-dimethyl-2,3,4,7-tetrahydroindolo[2,3-c]quinolin-1-one;
6-methyl-3′H-spirocyclohexane-2,3,4,7-tetrahydroindolo[2,3-c]quinolin-1-one;
10-chloro-3,3,6-trimethyl-2,3,4,7-tetrahydroindolo[2,3-c]quinolin-1-one;
10-methoxy-3,3-dimethyl-2,3,4,7-tetrahydroindolo[2,3-c]quinolin-1-one
11-fluoro-6-ethyl-3,3,-dimethyl-2,3,4,7-tetrahydroindolo[2,3-c]quinolin-1-one;
10-fluoro-6-ethyl-3,3,-dimethyl-2,3,4,7-tetrahydroindolo[2,3-c]quinolin-1-one;
3,3,-dimethyl-6-isopropyl-2,3,4,7-tetrahydroindolo[2,3-c]quinolin-1-one;
9-chloro-6-ethyl-3,3,-dimethyl-2,3,4,7-tetrahydroindolo[2,3-c]quinolin-1-one;
9-fluoro-6-ethyl-3,3,-dimethyl-2,3,4,7-tetrahydroindolo[2,3-c]quinolin-1-one;
10-fluoro-3-isopropyl-6-methyl-2,3,4,7-tetrahydroindolo[2,3-c]quinolin-1-one;
9-fluoro-3-isopropyl-6-methyl-2,3,4,7-tetrahydroindolo[2,3-c]quinolin-1-one;
11-fluoro-3-isopropyl-6-methyl-2,3,4,7-tetrahydroindolo[2,3-c]quinolin-1-one;
10-trifluoromethoxy-3,3,6-trimethyl-2,3,4,7-tetrahydroindolo[2,3-c]quinolin-1-one.

In certain embodiments, the compounds may be basic and form pharmaceutically acceptable salts with organic and inorganic acids.

Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalycilic acid, malic acid, fumaric acid, succinic acid, ascorbic acid, maleic acid, sulfonic acid, phosphonic acid, perchloric acid, nitric acid, propionic acid, gluconic acid, lactic acid, tartaric acid, hydroxymaleic acid, pyruvic acid, pnenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphersulfonic acid, china acid, mandelic acid, o-methylmandelic acid, hydrogen-benzenesulfonic acid, picric acid, adipic acid, D-o-tolyltartaric acid, tartronic acid, α-toluic acid (o, m, p), naphthylamine sulfonic acid, and other mineral or carboxylic acids well known to those skilled in the art. The salts may be prepared by contacting the free base form with a sufficient amount of the desired acid to produce a salt in the conventional manner.

The free base forms may be regenerated by treating the salt with a suitable dilute aqueous base solution, such as dilute aqueous sodium hydroxide, potassium carbonate, ammonia and sodium bicarbonate. The free base forms may differ from their corresponding salt forms in certain physical properties, such as solubility in polar solvents. The free base forms may differ from their corresponding salt forms in certain pharmacokinetic parameters, such as bioavailability, resulting in different pharmacological effects.

The present disclosure includes the pharmaceutically active free base forms of the compounds and pharmaceutically active salts of these compounds, all stereoisomeric forms and regioisomeric forms of these compounds or prodrugs thereof.

It is understood that certain embodiments of the present disclosure may have one or more chiral stereocenters. Such compounds may demonstrate biological activity as a racemic (or diastereomeric) mixture, as a mixture of R and S enantiomers (or diastereomers), or as pure enantiomers (R or S) (or diastereomers). When one pure enantiomer preferentially shows biological activity, this enantiomer is referred as the eutomer, whereas the less biologically active enantiomer is referred as the distomer.

By way of example, the compounds of Structure I wherein R¹ is lower alkyl, R⁴ and R⁵ together are oxygen, and R¹⁰ is hydrogen, can be prepared according to Scheme I, which involves a method of reacting an appropriate 2-(2-acyl-1H-indol-3-yl)-5,5-dimethyl-cyclohexane-1,3-dione with ammonium acetate in ethanol. A chemical synthesis of such compounds, as shown in Scheme I, discloses a novel approach to the synthesis of substituted 2,3,4,7-tetrahydroindolo[2,3-c]quinolin-1-ones.

More particularly, Scheme I involves a method of preparing an appropriate 1-oxo-2,3,4,7-tetrahydro-1H-5-oxonia-7-azabenzo[c]fluorene tetrafluoroborate, and, without isolation, reacting the 1-oxo-2,3,4,7-tetrahydro-1H-5-oxonia-7-azabenzo[c]fluorene tetrafluoroborate with water to form an appropriate 2-(2-acyl-1H-indol-3-yl)-5,5-dimethyl-cyclohexane-1,3-dione followed by reaction with ammonium acetate in ethanol to obtain an appropriate 2,3,4,7-tetrahydroindolo[2,3-c]quinolin-1-one. The use of an appropriate 2-(2-acyl-1H-indol-3-yl)-5,5-dimethyl-cyclohexane-1,3-dione instead of a 1-oxo-2,3,4,7-tetrahydro-1H-5-oxonia-7-azabenzo[c]fluorene perchlorate, and the direct conversion to the corresponding 2,3,4,7-tetrahydroindolo[2,3-c]quinolin-1-one improves the yield of substituted 2,3,4,7-tetrahydroindolo[2,3-c]quinolin-1-ones of Structure I by 25-40% over the process described in WO 2008/027182A2, which is hereby incorporated by reference. Scheme I provides a method for synthesizing substituted 2,3,4,7-tetrahydroindolo[2,3-c]quinolin-1-ones.

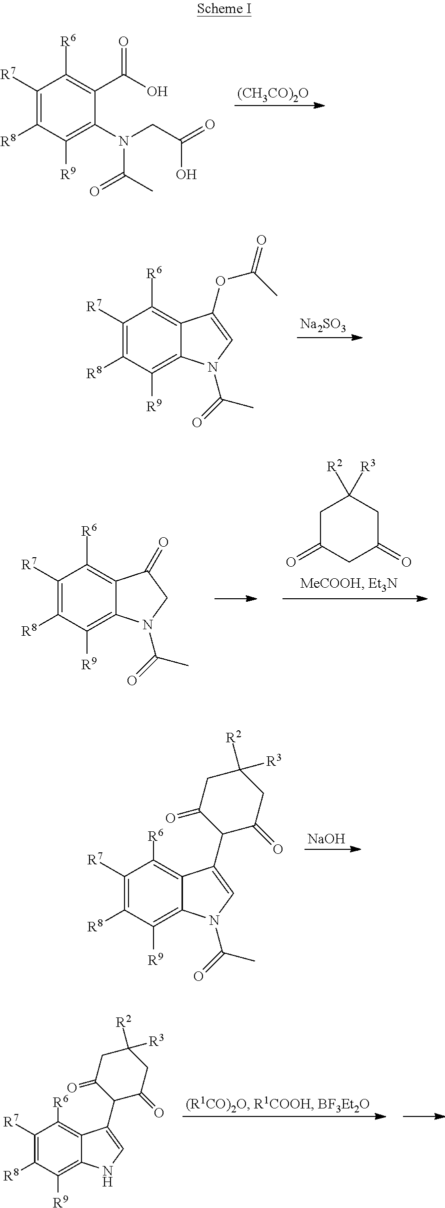

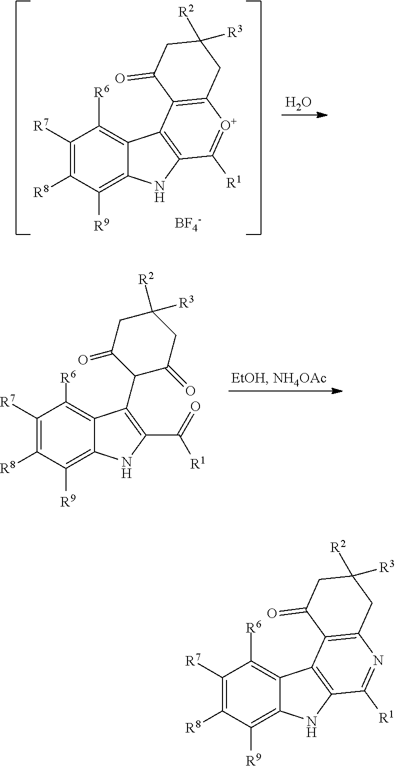

The chemical synthesis involves a method of making the substituted 2-(2-acyl-1H-indol-3-yl)-5,5-dimethyl-cyclohexane-1,3-dione intermediate of Structure II (shown below), by reacting 1-oxo-2,3,4,7-tetrahydro-1H-5-oxonia-7-azabenzo[c]fluorene tetrafluoroborates of Structure III without isolation with water. This is an improvement in the synthesis of compounds of this type.

In general, the 2-(N-acetylcarboxymethylamino)benzoic acid starting material shown in Scheme I can be prepared in two steps. First, an appropriate 2-aminobenzoic acid is reacted with chloroacetic acid using standard techniques (see, for example, S. Holt, et al., Proc. Roy. Soc. B 148: 481-494 (1958); S. Holt, et al., J. Chem. Soc. 1217-1223 (1958); E. Tighineanu, et al., Tetrahedron 36: 1385-1397 (1980); V. I. Dulenko, et al., Chem. Heterocycl. Comp. (Engl. Transl.) 3: 302-305 (1985); I. V. Komissarov, et al., Chem. Heterocycl. Comp. (Engl. Transl.) 19: 187-191 (1986); I. V. Komissarov, et al., Chem. Heterocycl. Comp. (Engl. Transl.) 23: 471-474 (1989); M. Kollmar, et al., Org. Synth. Coll. Vol. 10: 23 (2004)). Then, treatment of the substituted 2-(carboxymethylamino)benzoic acid with sodium carbonate and acetic anhydride provides a substituted 2-(N-acetylcarboxymethylamino)benzoic acid, which is the starting material shown in Scheme I.

Reaction of the substituted 2-(N-acetylcarboxymethylamino)benzoic acid starting material with acetic anhydride provides an acetic acid 1-acetyl-1H-indol-3-yl ester. This acetic acid 1-acetyl-1H-indol-3-yl ester can be reacted with sodium sulfite in water to provide a substituted 1-acetyl-1,2-dihydroindol-3-one. Reaction of the substituted 1-acetyl-1,2-dihydroindol-3-one with an appropriate cyclohexane-1,3-dione and triethylamine in acetic acid provides a 2-(1-acetyl-1H-indol-3-yl)cyclohexane-1,3-dione. This 2-(1-acetyl-1H-indol-3-yl)cyclohexane-1,3-dione may then be treated with sodium hydroxide to form a 2-(1H-indol-3-yl)cyclohexane-1,3-dione, Structure IV. Acylation of the 2-(1H-indol-3-yl)cyclohexane-1,3-dione with an appropriate carboxylic acid anhydride and boron trifluoride diethyl etherate provides the substituted 1-oxo-2,3,4,7-tetrahydro-1H-5-oxonia-7-azabenzo[c]fluorene tetrafluoroborate intermediate of Structure III. Without isolation, this intermediate is treated with water to provide the substituted 2-(2-acyl-1H-indol-3-yl)-5,5-dimethyl-cyclohexane-1,3-dione of Structure II. Treatment of the substituted 2-(2-acyl-1H-indol-3-yl)-5,5-dimethyl-cyclohexane-1,3-dione with ammonium acetate in ethanol provides the desired substituted 2,3,4,7-tetrahydroindolo[2,3-c]quinolin-1-one, Structure I.

Structures II, III and IV are generally:

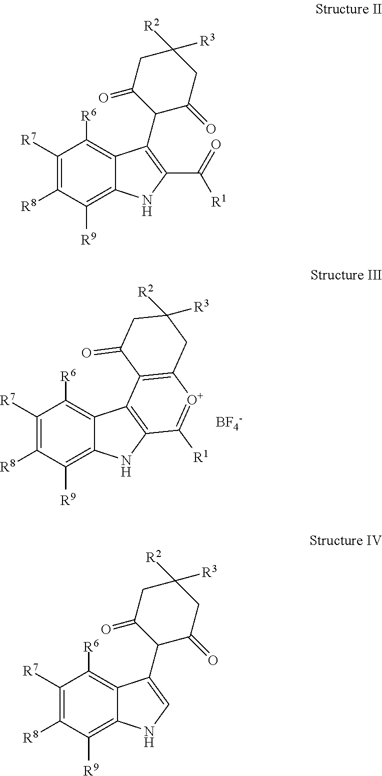

wherein:

R¹ is lower alkyl;

R² and R³ are independently one of: H, lower alkyl, cycloalk, aryl, arylalkyl; or R² and R³ are together —(CH₂)ₙ— and n is 6, 5 or 4; or R² and R³ are together —CH(lower alkyl)(CH₂)ₙ— and n is 5, 4 or 3. In certain embodiments, R² and R³ are independently one of: H, lower alkyl, aryl or arylalkyl; or R² and R³ are together —(CH₂)ₙ— and n is 6, 5 or 4. In certain embodiments, R² and R³ are independently one of: H, lower alkyl or aryl; or R² and R³ are together —(CH₂)ₙ— and n is 5 or 4; and

R⁶, R⁷, R⁸, and R⁹ are independently one of: H, halogen, CN, CF₃, OCF₃, lower alkyl, cycloalk, lower alkoxy, NH-lower alkyl, NH-alkylaryl, N(lower alkyl)₂, C(O)OH, C(O)O-lower alkyl, OH, OC(O)-lower alkyl. In certain embodiments, R⁶, R⁷, R⁸, and R⁹ are independently one of: H, halogen, CN, CF₃, OCF₃, lower alkyl, lower alkoxy.

In order to use a compound of Structure I or a pharmaceutically acceptable salt or complex thereof for the treatment of humans and other mammals, it is normally formulated in accordance with standard pharmaceutical practice as a pharmaceutical composition.

The compounds encompassed by Structure I may be administered by different routes including intravenous, intraperitoneal, subcutaneous, intramuscular, oral, topical (transdermal), or transmucosal administration. For systemic administration, oral administration is preferred. For oral administration, for example, the compounds encompassed by Structure I may be formulated into conventional oral dosage forms such as capsules, tablets, and liquid preparations such as syrups, elixirs, and concentrated drops.

Compositions of Structure I and their pharmaceutically acceptable salts and/or complexes, which are active when given orally, may be formulated as syrups, tablets, capsules, and lozenges. A syrup formulation will generally consist of a suspension or solution of the compound or salt in a liquid carrier such as, for example, ethanol, peanut oil, olive oil, glycerin or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule, any pharmaceutical carrier routinely used for preparing dispersions or suspensions may be utilized. For example, aqueous gums, celluloses, silicates or oils may be used to form a soft gelatin capsule shell.

Alternatively, injection (parenteral administration) may be used, e.g., intramuscular, intravenous, intraperitoneal, and subcutaneous. For injection, the compounds encompassed by Structure I may be formulated in liquid solutions, preferably, in physiologically compatible buffers or solutions, such as saline solution, Hank's solution, or Ringer's solution. Typical parenteral compositions consist of a solution or suspension of a compound or salt in a sterile aqueous or non-aqueous carrier optionally containing parenterally acceptable oil, for example polyethylene glycol, polyvinylpyrrolidone, lecithin, arachis oil or sesame oil. In addition, the compounds encompassed by Structure I may be formulated in solid form and redissolved or suspended immediately prior to use. Lyophilized forms can also be produced.

Typical compositions for inhalation are in the form of a solution, suspension or emulsion that may be administered as a dry powder or in the form of an aerosol using a conventional propellant such as dichlorodifluoromethane or trichlorofluoromethane.

Systemic administration can also be achieved by transmucosal or transdermal methods. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories. A typical suppository formulation comprises a compound of Structure I or a pharmaceutically acceptable salt or complex thereof which is active when administered in this way, with a binding and/or lubricating agent, for example polymeric glycols, gelatins, cocoa-butter or other low-melting vegetable waxes or fats or their synthetic analogs.

For topical administration, the compounds encompassed by Structure I may be formulated into ointments, salves, gels, or creams, as is generally known in the art. Typical dermal and transdermal formulations comprise a conventional aqueous or non-aqueous vehicle, for example a cream, ointment, lotion or paste or are in the form of a medicated plaster, patch or membrane.

The amounts of various compounds encompassed by Structure I to be administered can be determined by standard procedures taking into account factors such as the compound IC₅₀, EC₅₀, the biological half-life of the compound, the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses may have to be administered.

The composition may be in unit dosage form. For oral application, for example, a tablet or capsule may be administered; for nasal application, a metered aerosol dose may be administered; for transdermal application, a topical formulation or patch may be administered; and for transmucosal delivery, a buccal patch may be administered. In each case, dosing is such that the patient may administer a single dose.

Each dosage unit for oral administration may contain from 0.01 to 500 mg/kg, and in certain embodiments, from 0.1 to 50 mg/kg, of a compound of Structure I or a pharmaceutically acceptable salt or complex thereof, calculated as the free base. The daily dosage for parenteral, nasal, oral inhalation, transmucosal or transdermal routes may contain from 0.01 mg to 100 mg/kg of a compound of Structure I. A topical formulation may contain 0.01 to 5.0% of a compound of Structure I. The active ingredient may be administered as a single dose or in multiple doses, for example, from 2 to 6 times per day, sufficient to exhibit the desired activity, as is readily apparent to one skilled in the art.

As used herein, “treatment” of a disease or disorder includes, but is not limited to, alleviating at least one symptom of the disease or disorder, or delaying or suppressing the onset and/or development of the disease or disorder.

Diseases and disorders which might be treated or prevented, based upon the affected cells, include central nervous system diseases or disorders such as neurodegenerative diseases, and neurological disorders and diseases. As discussed above, alterations in the interaction of tau protein with microtubules and their stabilization have been identified in certain neurodegenerative diseases and disorders called tauopathies.

The term “neurodegenerative diseases” includes but is not limited to Down syndrome, Alzheimer's disease, Parkinson's disease, Huntington's disease, Pick's disease, Gerstmann-Sträussler-Scheinker disease with tangles, amyotrophic-lateral sclerosis, AIDS-related dementia, fragile X-associated tremor/ataxia syndrome (FXTAS), progressive supranuclear palsy (PSP), and striatonigral degeneration (SND), which is included with olivopontocerebellear degeneration (OPCD) and Shy Drager syndrome (SDS) in a syndrome known as multiple syndrome atrophy (MSA), brain injury, amyotrophic lateral sclerosis and inflammatory pain, regenerative (recovery) treatment of CNS disorders such as spinal cord injury, acute neuronal injury (stroke, traumatic brain injury), guam-parkinsonism-dementia complex, corticobasal neurodegeneration, frontotemporal dementia, mood disorders.

In one embodiment, the term “limited neurodegenerative diseases” includes Down syndrome, Alzheimer's disease, Parkinson's disease, Huntington's disease, Pick's disease, Gerstmann-Sträussler-Scheinker disease with tangles, fragile X-associated tremor/ataxia syndrome (FXTAS), progressive supranuclear palsy (PSP), and striatonigral degeneration (SND), which is included with olivopontocerebellear degeneration (OPCD) and Shy Drager syndrome (SDS) in a syndrome known as multiple syndrome atrophy (MSA).

The term “neurological disorders and diseases” includes but is not limited to adjustment disorders, anxiety disorders, delirium, dementia, amnestic and cognitive disorders, disorders usually first diagnosed in infancy, childhood, or adolescence, dissociative disorders (e.g. dissociative amnesia, depersonalization disorder, dissociative fugue and dissociative identity disorder), eating disorders, factitious disorders, impulse-control disorders, mental disorders due to general medical condition, mood disorders, other conditions that may be a focus of clinical attention, personality disorders, seizures, epilepsy, acute and chronic pain, schizophrenia and other psychotic disorders, sexual and gender identity disorders, sleep disorders, somatoform disorders, substance-related disorders, generalized anxiety disorder (e.g. acute stress disorder), panic disorder, phobia, agoraphobia, obsessive-compulsive disorder, stress, post-traumatic stress disorder, acute stress disorder, anxiety neurosis, nervousness, phobia, abuse, manic depressive psychosis, specific phobias, social phobia, adjustment disorder with anxious features.

In one embodiment, the term “limited neurological disorders and diseases” includes adjustment disorders, anxiety disorders, delirium, amnestic disorders, dissociative disorders (e.g. dissociative amnesia, depersonalization disorder, dissociative fugue and dissociative identity disorder), eating disorders, factitious disorders, impulse-control disorders, personality disorders, other psychotic disorders, sexual and gender identity disorders, sleep disorders, somatoform disorders, phobia, agoraphobia, specific phobias, social phobia, and adjustment disorder with anxious features.

No unacceptable toxicological effects are expected when compounds encompassed by Structure I are administered in accordance with the parameters described herein.

### EXAMPLES

The following specific examples are included for illustrative purposes only and are not to be considered as limiting to this disclosure. The reagents and intermediates used in the following examples are either commercially available or can be prepared according to standard literature procedures by those skilled in the art of organic synthesis.

NMR (Nuclear Magnetic Resonance) spectroscopy was performed on a Varian Gemini 300 spectrometer. Proton spectra were recorded at 300 MHz in deuterochloroform (CDCl₃), dimethylsulfoxide-d₆ (DMSO-d₆) or trifluoroacetic acid (CF₃COOH) solutions. NMR resonances are reported in δ (ppm) relative to tetramethylsilane (TMS) as internal standard with the following descriptors for the observed multiplicities: s (singlet), d (doublet), t (triplet), q (quartet), dd (doublet of doublets), and m (multiplet).

### General Procedures

### General Procedure A

### Preparation of the Compounds of Structure III

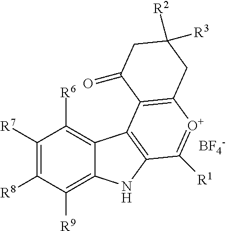

A compound of Structure IV (0.01 mol) was added to a mixture of an appropriate carboxylic acid (27 mL) and an appropriate carboxylic acid anhydride (0.027 mol) under stirring at room temperature. Boron trifluoride diethyl etherate (2.55 mL; 0.021 mol) was added dropwise. The reaction mixture immediately turned dark red, and a precipitate started to form. The mixture was stirred at room temperature for 2.5 h. Without isolation of the compound of Structure V, the reaction mixture was treated with water as described in General Procedure B.

### General Procedure B

### Preparation of the Compounds of Structure II

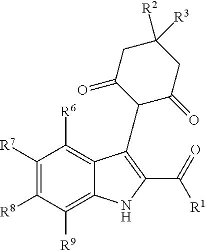

The reaction mixture of General Procedure A was treated with water (pre-cooled with ice, 300 mL), and the resultant mixture was stirred at room temperature overnight. The precipitate was collected, washed with water and air-dried to give 60-98% of the corresponding compound of Structure II. The product was used in the next step without purification.

### General Procedure C

### Preparation of the Compounds of Structure I

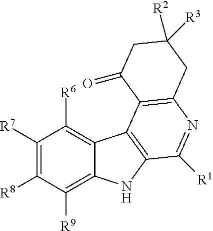

The compound of Structure II (0.0045 mol) was suspended in 92% aqueous ethanol (15.6 mL) with stirring, and ammonium acetate (2.16 g; 0.028 mol) was added. The mixture was refluxed for 2 h and evaporated under vacuum. The residue was treated with water (50 mL), and then 37% aqueous ammonia solution was added to bring the solution to pH 10. The precipitate was collected, washed with water, air-dried and dissolved in ethanol (40 mL). Aqueous 37% HCl (0.38 mL) was added to the ethanol solution, and the final solution was evaporated under vacuum to dryness. The residue was treated with acetone (40 mL), the precipitate was filtered off, washed with acetone, diethyl ether and air-dried to give the hydrochloride salt of the corresponding compound of Structure I. The hydrochloride salt was dissolved in hot water (95 mL), filtered off using a fritted glass filter (pore size 10-15 μm), and NH₄OH was added dropwise to the filtrate to a pH of 10. The precipitate was filtered off, washed with water and air-dried to give the compound of Structure I. The product was dissolved in ethyl acetate, the solution was filtered using a fritted glass filter (pore size 10-15 μm), and the filtrate was evaporated under vacuum to give the compound of Structure I with purity >98% in 75-96% yield.

### Example 1

### Preparation of 3,3,6-trimethyl-2,3,4,7-tetrahydroindolo[2,3-c]quinolin-1-one

### a) 2-(Carboxymethylamino)benzoic acid

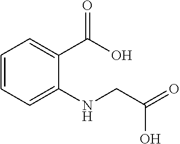

To a solution of chloroacetic acid (347 g, 3.67 mol) in water (500 mL), sodium carbonate (200 g, 1.88 mol) was carefully added at room temperature with stirring. The solution was heated to 40-45° C. and quickly added to a mixture prepared from a suspension of anthranilic acid (500 g, 3.65 mol) in water (340 mL) and 35% aqueous sodium hydroxide solution (320 mL) and heated to 40-45° C. The reaction mixture was kept at 40° C. for 4 days and the solid reaction mixture was treated with a solution of sodium hydroxide (150 g, 3.75 mol) in water (4 L). The mixture was heated to 60° C. and filtered while hot. The solid residue was washed on the filter with 20% aqueous sodium hydroxide until the solid residue was dissolved, and the combined filtrates were acidified with 37% aqueous hydrochloric acid to pH 3. The precipitate was filtered off; an additional amount of the product precipitated overnight from the filtrate and was collected. The product was dried at 100° C. to give total 567 g (80%), m.p. 220° C.

### b) 2-(N-Acetylcarboxymethylamino)benzoic acid

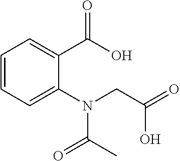

To a solution of sodium carbonate (89 g, 0.84 mol) in water (830 mL), 2-(carboxymethylamino)benzoic acid of Example 1a (100 g, 0.84 mol) was added in small portions at room temperature with stirring, forming a clear solution. Acetic anhydride (85.68 g, 0.84 mol) was added dropwise at room temperature with stirring. The reaction mixture was stirred for 30 min, and 37% aqueous hydrochloric acid (140 mL) was added dropwise. The product precipitated slowly. The solid product was filtered after 12 h, washed with water (3×150 mL) and air-dried to afford 110 g (91%), m.p. 206° C.

### c) Acetic acid 1-acetyl-1H-indol-3-yl ester

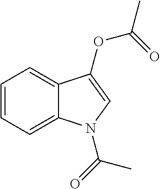

To a stirred, room-temperature mixture of acetic anhydride (46.29 g, 0.45 mol) and triethylamine (13.77 g, 0.14 mol), was added the 2-(N-acetylcarboxymethylamino)benzoic acid of Example 1b (11.36 g, 0.048 mol). The mixture was refluxed for 20 min and concentrated under vacuum to give an oily residue. Water (350 mL) was added with vigorous stirring, and the mixture was refrigerated overnight. The solid product was collected, washed with water and air-dried to give 9.0 g (86%) of the product, which was used in the next step without purification.

### d) 1-Acetyl-1,2-dihydroindol-3-one

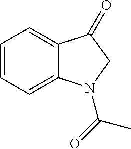

A solution of sodium sulfite (12.6 g, 0.1 mol) in water (180 mL) was heated to 70-75° C. under stirring, and the acetic acid 1-acetyl-1H-indol-3-yl ester of Example 1c (9.0 g, 0.041 mol) was added in small portions. The mixture was stirred at 70-75° C. for 1.5 h, and then kept at room temperature overnight. The solid product was filtered, dried, dissolved in methylene chloride (40 mL) and flash-chromatographed on aluminum oxide with methylene chloride as an eluent to give 5.25 g (71%) of light yellow product; m.p. 138° C.

### e) 2-(1-Acetyl-1H-indol-3-yl)-5,5-dimethylcyclohexane-1,3-dione

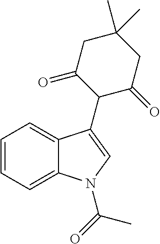

1-Acetyl-1,2-dihydroindol-3-one of Example 1d (131.3 g, 0.75 mol) and 5,5-dimethyl-cyclohexane-1,3-dione (105 g, 0.75 mol) were added to a mixture of acetic acid (700 mL) and triethylamine (105 mL, 0.75 mol) at room temperature with stirring. The reaction mixture was refluxed for 6 h. About one-third of the solvent volume was removed under vacuum, and the mixture was cooled and diluted with water (50 mL). The precipitate was filtered, washed with ethanol-water (1:1), and dried to afford 169.4 g (76%) of colorless crystals; m.p. 225-227° C.

### f) 2-(1H-Indol-3-yl)-5,5-dimethylcyclohexane-1,3-dione

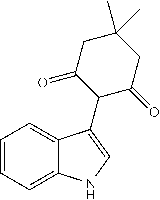

To a mixture of the 2-(1-acetyl-1H-indol-3-yl)-5,5-dimethylcyclohexane-1,3-dione of Example 1e (74.8 g, 0.25 mol) and methanol (30 mL), a solution of sodium hydroxide (30 g, 0.75 mol) in water (300 mL) was added at room temperature with stirring. The reaction mixture was heated at 60° C. for 2 h with stirring, and then activated charcoal (10 g) and water (300 mL) were added. The mixture was stirred for 10 min, the charcoal was filtered off, and the filtrate was acidified with hydrochloric acid to pH 2. The solid product was filtered, washed with water and dried to give 38.3 g (60%) of the colorless solid; m.p. 173° C.

### g) 3,3,6-Trimethyl-1-oxo-2,3,4,7-tetrahydro-1H-5-oxonia-7-azabenzo[c]fluorene tetrafluoroborate

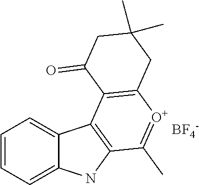

The title compound was prepared utilizing the procedures described in General Procedure A. Without isolation of the 3,3,6-trimethyl-1-oxo-2,3,4,7-tetrahydro-1H-5-oxonia-7-azabenzo[c]fluorene tetrafluoroborate, the next step involved formation of 2-(2-acetyl-1H-indol-3-yl)-5,5-dimethyl-cyclohexane-1,3-dione directly, as described in Example 1h.

### h) 2-(2-Acetyl-1H-indol-3-yl)-5,5-dimethyl-cyclohexane-1,3-dione

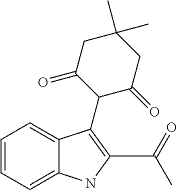

Utilizing the procedures described in General Procedure B, the title compound was prepared in 96% yield.

### i) 3,3,6-trimethyl-2,3,4,7-tetrahydroindolo[2,3-c]quinolin-1-one

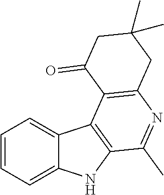

The 2-(2-Acetyl-1H-indol-3-yl)-5,5-dimethyl-cyclohexane-1,3-dione of Example 1h (0.0045 mol) was suspended in 92% aqueous ethanol (15.6 mL) with stirring, and ammonium acetate (2.16 g; 0.028 mol) was added. The mixture was refluxed for 2 h and evaporated under vacuum. The residue was treated with ethyl acetate (50 mL) and water (100 mL). Then the solution was basified with solid K₂CO₃ with stirring, to pH 10. The layers were separated, and the organic layer was washed with brine. The organic layer was dried over anhydrous Na₂SO₄ and evaporated under vacuum to give 3,3,6-trimethyl-2,3,4,7-tetrahydroindolo[2,3-c]quinolin-1-one as a light brown solid of 97.2% purity in 96% yield; m.p. 178.3-180.7° C. ¹H NMR (DMSO-d₆): δ 1.09 (s, 6H), 2.70 (s, 2H), 3.16 (s, 2H), 7.22-7.25 (m, 1H), 7.59-7.62 (m, 2H), 9.05 (s, 1H), 9.22 (d, 1H), 11.96 (s, 1H).

### Example 2

### Preparation of 11-fluoro-3,3,6-trimethyl-2,3,4,7-tetrahydroindolo[2,3-c]quinolin-1-one

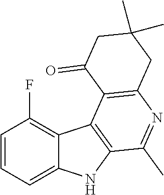

Utilizing the procedures described in Example 1 a-i except substituting 2-amino-6-fluorobenzoic acid for anthranilic acid in step 1a, the title compound was prepared in 73% yield; m.p. 246-247° C. (from toluene).

### Example 3

### Preparation of 9-fluoro-3,3,6-trimethyl-2,3,4,7-tetrahydroindolo[2,3-c]quinolin-1-one

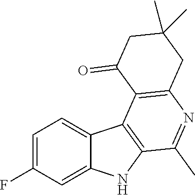

Utilizing the procedures described in Example 1 a-i except substituting 2-amino-4-fluorobenzoic acid for anthranilic acid in step 1a, the title compound was prepared in 94% yield; m.p. 244-246° C. (from toluene).

### Example 4

### Preparation of 10-fluoro-3,3,6-trimethyl-2,3,4,7-tetrahydroindolo[2,3-c]quinolin-1-one

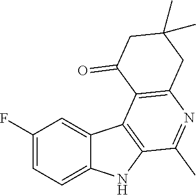

Utilizing the procedures described in Example 1 a-i except substituting 2-amino-5-fluorobenzoic acid for anthranilic acid in step 1a, the title compound was prepared in 92% yield; m.p. 229-230° C. (from toluene).

### Example 5

### Preparation of 3-isopropyl-6-methyl-2,3,4,7-tetrahydroindolo[2,3-c]quinolin-1-one

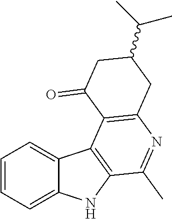

Utilizing the procedures described in Example 1 a-f except substituting 5-isopropyl-cyclohexane-1,3-dione for 5,5-dimethyl-cyclohexane-1,3-dione in step 1e, and then utilizing the procedures described for General Procedures A-C, the title compound was prepared in 55% yield; m.p. 248-249° C. (from toluene).

### Example 6

### Preparation of 10-methoxy-3,3,6-trimethyl-2,3,4,7-tetrahydroindolo[2,3-c]quinolin-1-one

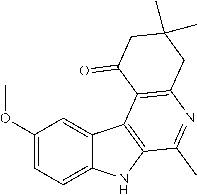

Utilizing the procedures described in Example 1 a-f except substituting 2-amino-5-methoxybenzoic acid for anthranilic acid in step 1a, and then utilizing the procedures described for General Procedures A-C, the title compound was prepared in 76% yield; m.p. 242-245° C. (from toluene).

### Example 7

### Preparation of 9-methoxy-3,3,6-trimethyl-2,3,4,7-tetrahydroindolo[2,3-c]quinolin-1-one

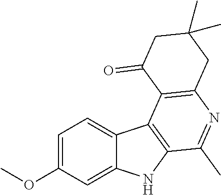

Utilizing the procedures described in Example 1 a-f except substituting 2-amino-4-methoxybenzoic acid for anthranilic acid in step 1a, and then utilizing the procedures described for General Procedures A-C, the title compound was prepared in 70% yield; m.p. 261-262° C. (from ethanol).

### Example 8

### Preparation of 3,6-dimethyl-2,3,4,7-tetrahydroindolo[2,3-c]quinolin-1-one

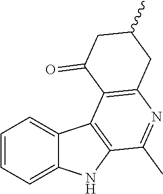

Utilizing the procedures described in Example 1 a-f except substituting 5-methyl-cyclohexane-1,3-dione for 5,5-dimethyl-cyclohexane-1,3-dione in step 1e, and then utilizing the procedures described for General Procedures A-C, the title compound was prepared in 55% yield; m.p. 218-219° C. (from toluene).

### Example 9

### Preparation of 10-fluoro-6-isopropyl-3,3,-dimethyl-2,3,4,7-tetrahydroindolo[2,3-c]quinolin-1-one

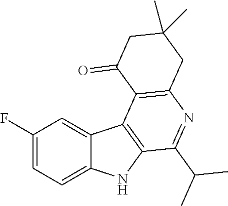

Utilizing the procedures described in Example 1 a-f except substituting 2-amino-5-fluorobenzoic acid for anthranilic acid in step 1a of Example 1, and then utilizing the procedures described for General Procedures A-C, the title compound was prepared in 75% yield; m.p. 182-183° C. (from toluene).

### Example 10

### Preparation of 6-ethyl-3,3-dimethyl-2,3,4,7-tetrahydro-indolo[2,3-c]quinolin-1-one

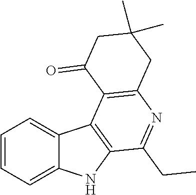

Utilizing the procedures described in Example 1 a-f, and then utilizing the procedures described for General Procedures A-C, the title compound was prepared in 77% yield; m.p. 188-190° C. (from toluene).

### Example 11

### Preparation of 3,3-dimethyl-2,3,4,7-tetrahydroindolo[2,3-c]quinolin-1-one

### a) 3,3-Dimethyl-1-oxo-2,3,4,7-tetrahydro-1H-5-oxonia-7-azabenzo[c]fluorene tetrafluoroborate

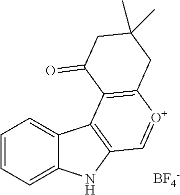

To a suspension of 2-(1H-indol-3-yl)-5,5-dimethylcyclohexane-1,3-dione of Example 1f (2.5 g, 9.8 mmol) in diethoxymethoxyethane (20 mL), tetrafluoroboric acid (54 wt % solution in diethyl ether, 2 mL, 14.5 mmol) was added in two portions under stirring. The mixture was stirred at room temperature for 2 h and diluted with diethyl ether (20 mL). The solid product was filtered off, washed with diethyl ether and air-dried at room temperature to give 2.4 g (70%) of the product as red crystals, which was used in the next step without purification.

### b) 3,3-Dimethyl-2,3,4,7-tetrahydroindolo[2,3-c]quinolin-1-one

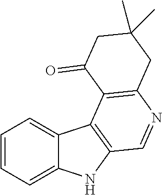

To a mixture of 3,3-dimethyl-1-oxo-2,3,4,7-tetrahydro-1H-5-oxonia-7-azabenzo[c]fluorene tetrafluoroborate of Example 11a (2.4 g; 6.8 mmol) and acetic acid (35 mL) was added ammonium acetate (30 g), and the mixture was refluxed for 30 min. The reaction mixture was cooled, water (40 mL) added, and then aqueous solution of ammonium hydroxide was added to pH 9. The solid product was filtered off, washed with water and air-dried at room temperature. The product was dissolved in the mixture of ethanol (15 mL) and diethyl ether (15 mL) at room temperature, and 37% aqueous hydrochloric acid (0.72 mL) was added dropwise. The mixture was kept at room temperature for 2 h. The crystalline product was filtered off, washed with ethanol-diethyl ether, 1:3 (2 mL), then with diethyl ether and dried to give 1.64 g of colorless crystals. This product was dissolved in water (30 mL) and aqueous ammonium hydroxide was added to pH 9. The precipitate was filtered off, washed with water, dried and crystallized from toluene to give 1.4 g (78%) of colorless crystals; m.p. 224-225° C. ¹H NMR (DMSO-d₆): δ 1.09 (s, 6H), 2.70 (s, 2H), 3.16 (s, 2H), 7.22-7.25 (m, 1H), 7.59-7.62 (m, 2H), 9.05 (s, 1H), 9.22 (d, 1H), 11.96 (s, 1H).

### Example 12

### Preparation of 6-methyl-3′H-spirocyclohexane-2,3,4,7-tetrahydroindolo[2,3-c]quinolin-1-one

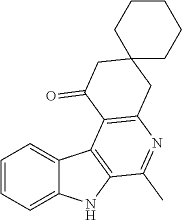

Utilizing the procedures described in Example 1 a-f, except substituting 3-(1H-indol-3-yl)-spiro[5.5]undecane-2,4-dione for 5,5-dimethyl-cyclohexane-1,3-dione in step 1e, and then utilizing the procedures described for General Procedures A-C, the title compound was prepared in 82% yield; m.p. 245-246° C. (from toluene).

### Example 13

### Preparation of 10-chloro-3,3,6-trimethyl-2,3,4,7-tetrahydroindolo[2,3-c]quinolin-1-one

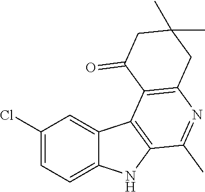

Utilizing the procedures described in Example 1 a-f except substituting 2-amino-5-chlorobenzoic acid for anthranilic acid in step 1a, and then utilizing the procedures described for General Procedures A-C, the title compound was prepared in 92% yield; m.p. 244-246° C. (from toluene).

### Example 14

### Preparation of 10-methoxy-3,3-dimethyl-2,3,4,7-tetrahydroindolo[2,3-c]quinolin-1-one

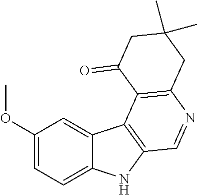

Utilizing the procedures described in Example 11 a,b except substituting 2-(6-methoxy-1H-indol-3-yl)-5,5-dimethyl-cyclohexane-1,3-dione for 2-(1H-indol-3-yl)-5,5-dimethylcyclohexane-1,3-dione in step 11a and 2-amino-5-methoxybenzoic acid for anthranilic acid in step 1a of Example 1, the title compound was prepared in 89% yield; m.p. 230-231° C. (from toluene).

### Example 15

### Preparation of 11-fluoro-6-ethyl-3,3,-dimethyl-2,3,4,7-tetrahydroindolo[2,3-c]quinolin-1-one

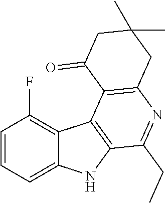

Utilizing the procedures described in Example 1 a-f except substituting 2-amino-6-fluorobenzoic acid for anthranilic acid in step 1a, and then utilizing the procedures described for General Procedures A-C, the title compound was prepared in 66% yield; m.p. 237-240° C. (from toluene-hexane).

### Example 16

### Preparation of 10-fluoro-6-ethyl-3,3,-dimethyl-2,3,4,7-tetrahydroindolo[2,3-c]quinolin-1-one

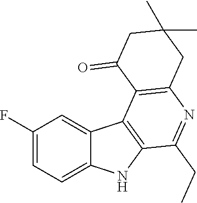

Utilizing the procedures described in Example 1 a-f except substituting 2-amino-5-fluorobenzoic acid for anthranilic acid in step 1a, and then utilizing the procedures described for General Procedures A-C, the title compound was prepared in 66% yield; m.p. 192-194° C. (from toluene-hexane).

### Example 17

### Preparation of 3,3,-dimethyl-6-isopropyl-2,3,4,7-tetrahydroindolo[2,3-c]quinolin-1-one

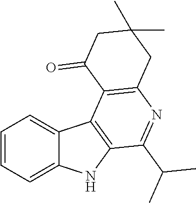

Utilizing the procedures described in Example 1 a-f, and then utilizing the procedures described for General Procedures A-C, the title compound was prepared in 68% yield; m.p. 182-184° C. (from toluene-hexane).

### Example 18

### Preparation of 9-chloro-6-ethyl-3,3,-dimethyl-2,3,4,7-tetrahydroindolo[2,3-c]quinolin-1-one

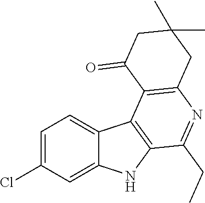

Utilizing the procedures described in Example 1 a-f except substituting 2-amino-4-chlorobenzoic acid for anthranilic acid in step 1a, and then utilizing the procedures described for General Procedures A-C, the title compound was prepared in 75% yield; m.p. 198-200° C. (from toluene-hexane).

### Example 19

### Preparation of 9-fluoro-6-ethyl-3,3,-dimethyl-2,3,4,7-tetrahydroindolo[2,3-c]quinolin-1-one

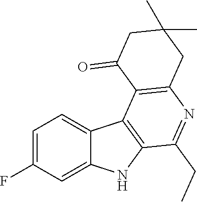

Utilizing the procedures described in Example 1 a-f except substituting 2-amino-4-fluorobenzoic acid for anthranilic acid in step 1a, and then utilizing the procedures described for General Procedures A-C, the title compound was prepared in 65% yield; m.p. 180-182° C. (from toluene-hexane).

### Example 20

### Preparation of 10-fluoro-3-isopropyl-6-methyl-2,3,4,7-tetrahydroindolo[2,3-c]quinolin-1-one

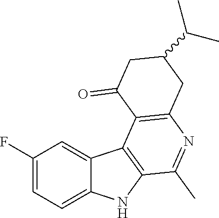

Utilizing the procedures described in Example 1 a-f except substituting -amino-5-fluorobenzoic acid for anthranilic acid in step 1a and 5-isopropyl-cyclohexane-1,3-dione for 5,5-dimethyl-cyclohexane-1,3-dione in step 1e, and then utilizing the procedures described for General Procedures A-C, the title compound was prepared in 58% yield; m.p. 221-223° C. (from toluene).

### Example 21

### Preparation of 9-fluoro-3-isopropyl-6-methyl-2,3,4,7-tetrahydroindolo[2,3-c]quinolin-1-one

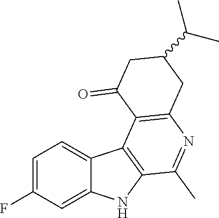

Utilizing the procedures described in Example 1 a-f except substituting -amino-4-fluorobenzoic acid for anthranilic acid in step 1a and 5-isopropyl-cyclohexane-1,3-dione for 5,5-dimethyl-cyclohexane-1,3-dione in step 1e, and then utilizing the procedures described for General Procedures A-C, the title compound was prepared in 66% yield; m.p. 285-286° C. (from toluene).

### Example 22

### Preparation of 11-fluoro-3-isopropyl-6-methyl-2,3,4,7-tetrahydroindolo[2,3-c]quinolin-1-one

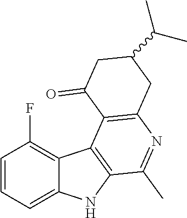

Utilizing the procedures described in Example 1 a-f except substituting -amino-6-fluorobenzoic acid for anthranilic acid in step 1a and 5-isopropyl-cyclohexane-1,3-dione for 5,5-dimethyl-cyclohexane-1,3-dione in step 1e, and then utilizing the procedures described for General Procedures A-C, the title compound was prepared in 59% yield; m.p. 213-215° C. (from toluene).

### Example 23

### Preparation of 10-trifluoromethoxy-3,3,6-trimethyl-2,3,4,7-tetrahydroindolo[2,3-c]quinolin-1-one

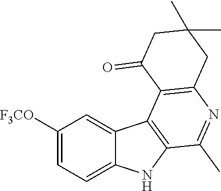

Utilizing the procedures described in Example 1 a-f except substituting 2-amino-5-trifluoromethoxybenzoic acid for anthranilic acid in step 1a, and then utilizing the procedures described for General Procedures A-C, the title compound was prepared in 96% yield; m.p. 179-170° C. (from toluene).

The biological activities of certain embodiments of the compounds of Structure I were evaluated using in vitro kinase binding and functional assays, an assay in a cultured cell line, and a study of pharmacokinetic parameters in rats.

(I) Human DYRK1A Kinase Binding Assay and Binding Constant Measurements.

Human DYRK1A kinase profiles in a competitive binding assay were performed by Ambit Biosciences (San Diego, Calif., USA). Activity was recorded via a competition binding assay of human DYRK1A kinase that was fused to a proprietary tag. Measurements of the amount of DYRK1A kinase bound to an immobilized, active-site directed ligand in the presence and absence of the test compound provided a percentage of DMSO control for binding of ligand. Activities between 1% and 10% were selected for Kd determination. Dendrogram representations were generated by an in-house visualization tool designated as PhyloChem.

In certain embodiments, compounds have a low Kd value in the DYRK1A kinase binding assay. Compounds useful in certain embodiments of the current disclosure have Kd values below about 50 uM. In another embodiment, compounds have a Kd of about 10 uM or lower. In another embodiment, compounds have a Kd of about 5 uM or lower. In a further embodiment, compounds have a Kd of about 1 uM or lower. The human DYRK1A kinase binding assay has been used to test the compounds of the Examples disclosed. Examples 1, 2, 4 and 6 have Kd values below about 10 uM, Example 6 has Kd value below about 5 uM, and Examples 1, 2 and 4 have Kd values below about 1 uM in this assay.

(II) Human DYRK1A Kinase Functional Assay and IC₅₀ Measurements.

Compound profiling in a human DYRK1A kinase functional assay was performed by CEREP (France). The effects of compounds on the activity of human DYRK1A were evaluated and quantified by measuring the phosphorylation of the substrate Ulight-CFFKNIVTPRTPPPSQGK-amide (MBP) using a human recombinant enzyme and the LANCE® detection method, as described by Himpel et al, J. Biol. Chem., 275: 2431 (2000). The inhibitory activity of the compounds was expressed as a percent inhibition of the control kinase activity. Activities of the compounds at 50% or higher inhibition were selected for IC₅₀ determination. Compounds were tested at 6 concentrations in duplicate for IC₅₀ determination. The standard inhibitory reference compound was staurosporine, which was tested in each experiment at 8 concentrations to obtain an inhibition curve from which its IC₅₀ value was calculated.

In certain embodiments, compounds have a low IC₅₀ value in the DYRK1A kinase functional assay. Compounds useful in certain embodiments of the current disclosure have IC₅₀ values below about 50 uM. In another embodiment, compounds have a IC₅₀ of about 10 uM or lower. In another embodiment, compounds have a IC₅₀ of about 5 uM or lower. In a further embodiment, compounds have a IC₅₀ of about 2 uM or lower. The human DYRK1A kinase functional assay has been used to test the compounds of the Examples disclosed. Examples 12 and 14 have IC₅₀ values below about 10 uM, Examples 2, 8, 10, 11, 13 and 15 have IC₅₀ values below about 5 uM, and Examples 1, 4 and 16 have IC₅₀ values below about 2 uM in this assay.

(III) Human H4 Neuroglioma Cell Line Assay of Tau Phosphorylation.

Compound activity in a cultured human H4 neuroglioma cell line, monitoring the inhibition of tau phosphorylation following inhibition of DYRK1A kinase, was performed by Translational Genomics Research Institute (Phoenix, Ariz.). The effects of compounds on the reduction of human tau phosphorylation at the Ser262/Ser356 (12E8), Thr231 and Ser396 sites were evaluated in a human H4 neuroglioma cell line. Ser262/Ser356 (12E8) is an early site of hyperphosphorylation that controls tau's microtubule affinity; Thr231 is another microtubule affinity-regulating site, but is hyperphosphorylated concomitant to tangle formation; and Ser396 is a site of phosphorylation occurring in late stages of tangle formation. The activity of the compounds was quantified by measuring the expression of pS262/pS356, pT231, and pS396 by Western blot analyses from cells treated with either vehicle or the compounds.

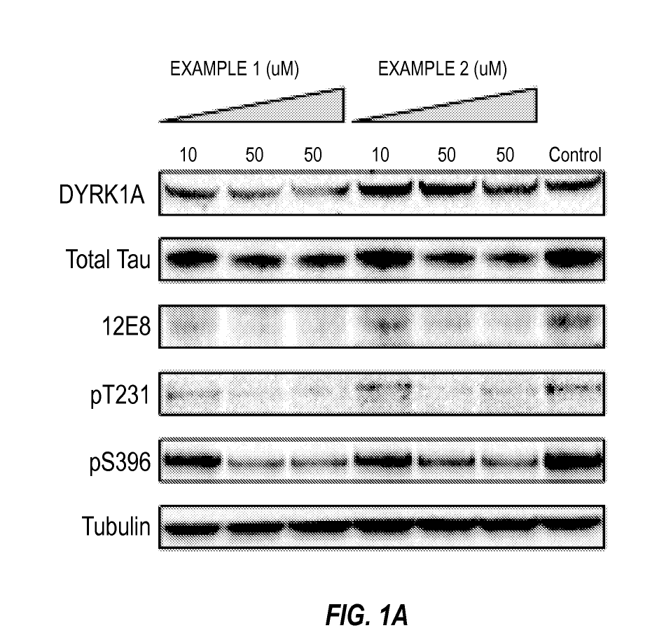

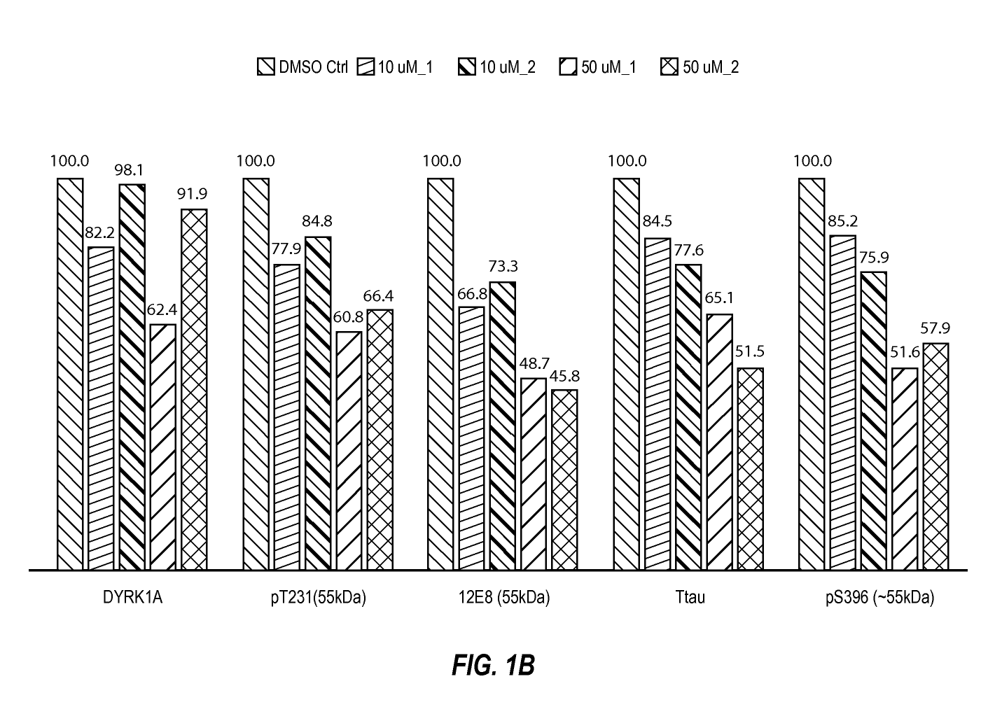

In certain embodiments, compounds effectively reduce the expression of phosphorylated forms of tau protein after a 96-hour treatment at concentrations of 10 to >100 uM lower than their concentrations of measured cytotoxicity. Compounds useful in certain embodiments of the current disclosure reduce the expression of pS262/pS356, pT231, pS396 and total tau protein by 20% or more. In another embodiment, compounds reduce the expression of pS262/pS356, pT231, pS396 and total tau protein by 30% or more. In a further embodiment, compounds reduce the expression of pS262/pS356, pT231 and pS396 and total phosphorylated tau protein by 40% or more. The human H4 neuroglioma cell line has been used to test the compounds of the Examples disclosed. The compounds of Examples 1 and 2 have been tested in human H4 neuroglioma cell line at 10 uM and 50 uM concentration and reduced the expression of pS262/pS356, pT231, pS396 by 20 to 50% as shown in FIG. 1.

(IV) Pharmacokinetic Studies

The pharmacokinetic studies in Sprague-Dawley rats were performed using the protocols developed by WuXi AppTec (St. Paul, Minn., USA, and Shanghai, China. http://www.apptecls.com/). The pharmacokinetic parameters of the compound of Example 1 are shown in Table 1.

TABLE 1
Plasma PK parameters of the compound of Example 1 at
a single dose administration in normal rats (n = 3)

| | Dose, mg/kg | | |
|---|---|---|---|
| Pharmacokinetic | | i.v. | p.o. |

| parameters | 2 | 10 | 10 |
|---|---|---|---|
| Cₒ (ng/ml) | 1665 | 4100 | — |
| Cₘₐₓ (ng/mL | — | — | 1865 |
| t_{1/2} (h) | 0.370 | 0.765 | 0.821 |
| Tₘₐₓ (h) | — | — | 0.25 |
| Vdss (L/kg) | 2.31 | 3.81 | — |
| Vdss₀₋ₗₐₛₜ (L/kg) | — | 3.39 | — |
| CL (mL/min/kg) | 106 | 71.1 | — |
| CL₀₋ₗₐₛₜ (mL/min/kg) | — | 73.1 | — |
| AUC₀₋ₗₐₛₜ (ng · h/mL) | 312 | 2280 | 541 |
| AUC_{0-inf} (ng · h/mL) | 316 | 2350 | 544 |
| AUC_{Extra} (%) | 1.49 | — | 0.672 |
| MRT₀₋ₗₐₛₜ (h) | 0.332 | 0.773 | 0.610 |
| MRT_{0-inf} (h) | 0.365 | 0.893 | 0.655 |
| F* (%) | — | — | 34 |

*Absolute oral bioavailability is calculated using AUC_{0-inf}.

The brain-plasma pharmacokinetic parameters of the compound of Example 1 are shown in Table 2. The brain exposure of the compound of Example 1 is about 30- to 4-fold higher than that required for inhibition of DYRK1A, with a Kd and IC₅₀ at about 1 uM, or 280 ng/mL.

TABLE 2
Brain/Plasma ratio for the compound of Example 1 at a single
dose of 10 mg/kg bolus i.v. injection in normal rats (n = 3)

| | Mean concentration | Mean concentration | Brain/ | AUC₀₋ₜ of | AUC₀₋ₜ of | B_{AUC}/ |
|---|---|---|---|---|---|---|
| Time | in plasma | in brain | Plasma | Plasma | Brain | P_{AUC} |
| (h) | (ng/mL) | (ng/mL) | Ratio | (ng · h/mL) | (ng · h/g) | Ratio^{a} |
| 0.25 | 2392 | 6905 | 2.89 | 792 | 2409 | 3.04 |
| 1 | 476 | 1033 | 2.17 | 1682 | 4727 | 2.81 |
| 4 | 58.7 | 99.4 | 1.69 | 2280 | 5924 | 2.60 |
| 8 | — | — | — | 2350^{b} | 6020^{b} | 2.56 |
| 24 | — | — | — | 2350^{b} | 6020^{b} | 2.56 |

^{a}B_{AUC}/P_{AUC} Ratio = BrainAUC₍₀₋ₜ₎/PlasmaAUC₍₀₋ₜ₎.
^{b}The data is the value of AUC_{(inf)}.

The above description fully discloses the invention including certain embodiments thereof. Modifications and improvements of the embodiments specifically disclosed herein are within the scope of the following claims. Without further elaboration, it is believed that one skilled in the area can, using the preceding description, utilize the present invention to its fullest extent. The Examples are not intended to limit the present disclosure, although the specifics recited herein may include independently patentable subject matter.

It will be obvious to those having skill in the art that many changes may be made to the details of the above-described embodiments without departing from the underlying principles of the invention. The scope of the present invention should, therefore, be determined only by the following claims.

1. A compound according to Structure I, 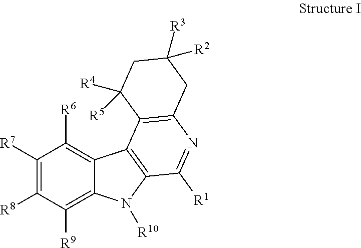
wherein:
R¹ is one of: H, alkyl, cycloalk, aryl, arylalkyl;
R² and R³ are each independently selected from one of: H, alkyl, cycloalk, aryl, arylalkyl; or R² and R³ are together —(CH₂)ₙ— and n is 6, 5 or 4; or R² and R³ are together —CH(lower alkyl)(CH₂)ₙ— and n is 5, 4 or 3;
R⁴ and R⁵ are each independently selected from one of: H, NH₂, OH or lower alk; or R⁴ and R⁵ are together O, S or NOH;
R⁶, R⁸, and R⁹ are each independently selected from the group consisting of:
H, halogen, CN, CF₃, OCF₃, alkyl, cycloalk, lower alkoxy, NH-lower alk, NH-alkylaryl, N(lower alkyl)₂, C(O)OH, C(O)O-lower alk, OH, OC(O)-lower alkyl;
R⁷ is haloalkoxy, and
R¹⁰ is one of: H, alkyl, cycloalk, aryl, arylalkyl;
and pharmaceutically acceptable hydrates, solvates, tautomers, and complexes thereof.

2. A compound according to claim 1 wherein:
R¹ is methyl;
R² and R³ are each methyl;
R⁴ and R⁵ are together O;
R⁶, R⁸, and R⁹ are each H;
R⁷ is lower alkoxy; and
R¹⁰ is H.

3. A compound according to Structure I, 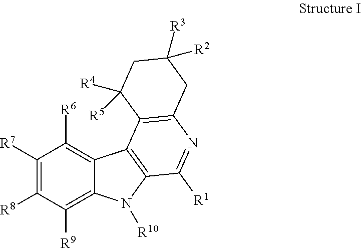
wherein:
R¹ is one of: H, lower alk, cycloalk, aryl, arylalkyl;
one of R² and R³ is lower alkyl and the other is H;
or R² and R³ are together —(CH₂)ₙ— and n is 6, 5 or 4;
R⁴ and R⁵ are each independently selected from one of: H, NH₂, OH or lower alk;
or R⁴ and R⁵ are together O, S or NOH;
R⁶, R⁷, R⁸, and R⁹ are each H; and
R¹⁰ is one of: H, lower alkyl, cycloalk;
and pharmaceutically acceptable hydrates, solvates, tautomers, and complexes thereof.

4. A compound according to claim 3 wherein:
R¹ is methyl;
one of R² and R³ is lower alkyl and the other is H;
R⁴ and R⁵ are together O; and
R¹⁰ is H.

5. A compound according to claim 3 wherein:
R¹ is methyl;
R² and R³ are together —(CH₂)ₙ— and n is 6, 5, or 4;
R⁴ and R⁵ are together O; and
R¹⁰ is H.

6. A compound according to Structure I, 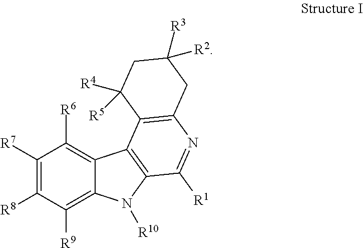
wherein:
R¹ is one of H or alkyl, with the proviso that R¹ is not methyl;
R² and R³ are each independently lower alk;
R⁴ and R⁵ are each independently selected from one of: H, NH₂, OH or lower alk; or R⁴ and R⁵ are together O, S or NOH;
R⁶, R⁷, R⁸, and R⁹ are each H; and
R¹⁰ is one of: H, lower alkyl, cycloalk, aryl, arylalkyl;
and pharmaceutically acceptable hydrates, solvates, tautomers, and complexes thereof.

7. A compound according to claim 6 wherein:
R¹ is C2-C6 alkyl;
R² and R³ are each methyl;
R⁴ and R⁵ are together O;
R⁶, R⁷, R⁸, and R⁹ are each H; and
R¹⁰ is H.

8. A compound according to claim 6 wherein:
R¹ is H;
R² and R³ are each methyl;
R⁴ and R⁵ are together O;
R⁶, R⁷, R⁸, and R⁹ are each H;
and R¹⁰ is H.

9. A compound according to claim 6 wherein:
R¹ is lower alkyl;
R² and R³ are each methyl;
R⁴ and R⁵ are together O;
R⁶, R⁷, R⁸, and R⁹ are each H; and
R¹⁰ is H.

11. A method for preparing substituted 2,3,4,7-tetrahydroindolo[2,3-c]quinolines, comprising:
reacting substituted 2-(N-acetylcarboxymethylamino)benzoic acids with acetic anhydride to provide an acetic acid 1-acetyl-1H-indol-3-yl ester;
reacting the acetic acid 1-acetyl-1H-indol-3-yl ester with sodium sulfite to provide a 1-acetyl-1,2-dihydroindol-3-one;
reacting the 1-acetyl-1,2-dihydroindol-3-one with a cyclohexane-1,3-dione and triethylamine to provide a 2-(1-acetyl-1H-indol-3-yl)cyclohexane-1,3-dione;
reacting the 2-(1-acetyl-1H-indol-3-yl)cyclohexane-1,3-dione with sodium hydroxide to provide a 2-(1H-indol-3-yl)cyclohexane-1,3-dione;
acylating the 2-(1H-indol-3-yl)cyclohexane-1,3-dione to provide a 1-oxo-2,3,4,7-tetrahydro-1H-5-oxonia-7-azabenzo[c]fluorene tetrafluoroborate;
reacting the 1-oxo-2,3,4,7-tetrahydro-1H-5-oxonia-7-azabenzo[c]fluorene tetrafluoroborate without isolation with water to provide a 2-(2-acyl-1H-indol-3-yl)-5,5-dimethyl-cyclohexane-1,3-dione;
and reacting the 2-(2-acyl-1H-indol-3-yl)-5,5-dimethyl-cyclohexane-1,3-dione with ammonium acetate to provide the substituted 2,3,4,7-tetrahydroindolo[2,3-c]quinolin-1-one.

12. A compound comprising:
10-fluoro-3,3-dimethyl-6-isopropyl-2,3,4,7-tetrahydroindolo[2,3-c]quinolin-1-one.
11-fluoro-6-ethyl-3,3,-dimethyl-2,3,4,7-tetrahydroindolo[2,3-c]quinolin-1-one;
10-fluoro-6-ethyl-3,3,-dimethyl-2,3,4,7-tetrahydroindolo[2,3-c]quinolin-1-one;
3,3,-dimethyl-6-isopropyl-2,3,4,7-tetrahydroindolo[2,3-c]quinolin-1-one;
9-chloro-6-ethyl-3,3,-dimethyl-2,3,4,7-tetrahydroindolo[2,3-c]quinolin-1-one;
9-fluoro-6-ethyl-3,3,-dimethyl-2,3,4,7-tetrahydroindolo[2,3-c]quinolin-1-one;
10-fluoro-3-isopropyl-6-methyl-2,3,4,7-tetrahydroindolo[2,3-c]quinolin-1-one;
9-fluoro-3-isopropyl-6-methyl-2,3,4,7-tetrahydroindolo[2,3-c]quinolin-1-one;
11-fluoro-3-isopropyl-6-methyl-2,3,4,7-tetrahydroindolo[2,3-c]quinolin-1-one;
10-trifluoromethoxy-3,3,6-trimethyl-2,3,4,7-tetrahydroindolo[2,3-c]quinolin-1-one.
